(19) United States
(12) Patent Application Publication
(10) Pub. No.: US 2009/0098219 A1
(43) Pub. Date: Apr. 16, 2009

(54) Cosmetic/pharmaceutical compositions comprising retinoids and anti-irritants and treatment of keratinization disorders therewith

(75) Inventors: Laurent Fredon, Roquefort Les Pins (FR); Claire Mallard, Mougins (FR); Eve Ferrara, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/213,154

(22) Filed: Jun. 16, 2008

(30) Foreign Application Priority Data
Dec. 15, 2005 (FR) 0512759
Nov. 28, 2006 (FR) PCT/FR2006/051243

(51) Int. Cl. A61K 31/192; A61K 8/368; A61K 31/4166; A61P 17/00

(52) U.S. Cl. 424/718; 514/569; 514/390; 424/722

(57) ABSTRACT

Topically applicable cosmetic/pharmaceutical compositions contain at least one retinoid and at least one anti-irritant compound selected from among is allantoin, divalent strontium salts, divalent zinc salts, monovalent sodium salts and the hydrated derivatives thereof, and are useful for the treatment and/or prevention of a dermatological condition or affliction related to a disorder of keratinization relating to cell differentiation and to cell proliferation, e.g. acne vulgaris.

### CROSS-REFERENCE TO COMPANION APPLICATION

Copending U.S. Patent Application No. ______ [Attorney Docket No. 1034227-000927], filed concurrently herewith, hereby expressly incorporated by reference and also assigned to the assignee hereof.

### CROSS-REFERENCE TO PRIORITY/PCT APPLICATIONS

This application claims priority under 35 U.S.C. § 119 of FR 0512759, filed Dec. 15, 2005, and is a continuation of PCT/FR 2006/051243, filed Nov. 28, 2006, and designating the United States (published in the French language on Jun. 28, 2007, as WO 2007/071861 A2; the title and abstract were also published in English), each hereby expressly incorporated by reference in its entirety and each assigned to the assignee hereof.

### BACKGROUND OF THE INVENTION

1. Technical Field of the Invention

The present invention relates to compositions for topical administration and to their applications as cosmetic or pharmaceutical products, such compositions being useful, in particular, for the treatment of acne.

2. Description of Background and/or Related and/or Prior Art

Acne is a common multifactor pathology which affects skin rich in sebaceous glands (face, scapula region, arms and inter-triginous regions). It is the commonest form of dermatosis. The following five pathogenic factors play a determining role in the formation of acne:

1. genetic predisposition;

2. overproduction of sebum (seborrhea);

3. androgens;

4. follicular keratinization disorders (comedogenesis); and

5. bacterial colonization and inflammatory factors.

Several forms of acne exist, all having in common that the pilosebaceous follicles are attacked. Exemplary are acne conglobata, acne keloid on the back of the neck, acne medicamentosa, recurrent acne miliaria, acne necrotica, acne neonatorum, premenstrual acne, occupational acne, acne rosacea, senile acne, solar acne and acne vulgaris.

Acne vulgaris, also known as polymorphous juvenile acne, is the commonest. It comprises four stages:

Stage 1 corresponds to comedonal acne, characterized by a large number of open and/or closed comedones and of microcysts.

Stage 2, or papulopustular acne, is of mild to moderate seriousness. It is characterized by the presence of open and/or closed comedones and of microcysts but also of red papules and of pustules. It mainly affects the face and leaves few scars.

Stage 3, or papulocomedonal acne, is more serious and extends to the back, to the thorax and to the shoulders. It is accompanied by a larger number of scars.

Stage 4, or nodulocystic acne, is accompanied by numerous scars. It exhibits nodules and also large painful purplish pustules.

The various forms of acne described above can be treated with active principles, such as anti-seborrheics and anti-infectives, for example benzoperoxide (in particular, the product Eclaran® marketed by Pierre Fabre), with retinoids, such as tretinoin (in particular, the product Retacnyl® marketed by Galderma) or isotretinoin (product Roaccutane® marketed by Laboratoires Roche), or with napthoic acid derivatives. Naphthoic acid derivatives, such as, in particular, 6-[3-(1-adamantyl)4-methoxyphenyl]-2-naphthoic acid, commonly known as adapalene (the product Differin® marketed by Galderma), are widely described and recognized as active principles which are as effective as tretinoin in the treatment of acne.

Adapalene in particular, exhibits an effectiveness which is accepted by all; however, it would be advantageous and useful for its tolerance by the topical route, although better than that of its competitors belonging to the same chemical category (tretinoin, tazarotene), to be improved.

### SUMMARY OF THE INVENTION

It has now surprisingly been discovered that the combination of adapalene with certain specific anti-irritant compounds significantly improves the tolerance of this retinoid and thus overcomes the problem of irritation. This is because, as shown in the Example 2 to follow, certain anti-irritants reduce by up to 40% the edema caused by adapalene.

The present invention thus features compositions, in particular pharmaceutical compositions and preferably dermatological compositions, intended in particular for topical application, comprising, formulated into a physiologically acceptable medium, at least one retinoid compound, preferably selected from among the naphthoic acid derivatives of formula (I) below, their salts and their esters, and at least one anti-irritant compound selected from among allantoin, EDTA, divalent strontium salts, divalent zinc salts, monovalent sodium salts, and the hydrated derivatives thereof. Preferably, the subject compositions do not comprise any depigmenting agent.

In particular, such compositions do not comprise any depigmenting agent other than the retinoid compound, in particular, adapalene.

The term “physiologically acceptable medium” means a medium compatible with the skin, mucous membranes and/or superficial body growths.

### BRIEF DESCRIPTION OF THE DRAWINGS

FIG. 1 is a graph comparing the irritant power of reference gels versus that according to the present invention;

FIG. 2 is a graph comparing the irritant power of reference gels versus that according to the present invention;

FIG. 3 is a graph comparing the irritant power of reference gels versus that according to the present invention;

FIG. 4 is a histograph of the AUC for edemas D2-D19, and

FIG. 5 is a graph showing the number of comedones on the back of Rhino mice after 18 days of topical treatment with the gels of FIGS. 1-3.

### DETAILED DESCRIPTION OF BEST MODE AND SPECIFIC/PREFERRED EMBODIMENTS OF THE INVENTION

The retinoid compounds according to the invention can be selected from among all trans retinoic acid (or tretinoin), isotretinoin or motretinide.

The retinoid compounds according to the invention are preferably selected from among naphthoic acid derivatives of formula (I), the salts and the esters thereof:

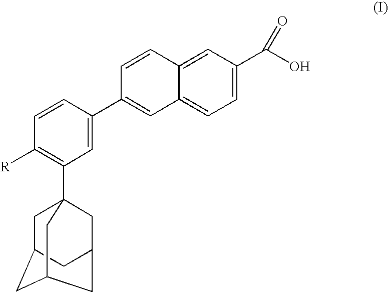

wherein R is a hydrogen atom, a hydroxyl radical, a linear or branched alkyl radical having from 1 to 4 carbon atoms, an alkoxy radical having from 1 to 10 carbon atoms or a cycloaliphatic radical which is unsubstituted or substituted.

The term “linear or branched alkyl radical having from 1 to 4 carbon atoms” means, preferably, the methyl, ethyl, propyl and butyl radicals.

The term “alkoxy radical having from 1 to 10 carbon atoms” means, preferably, the methoxy, ethoxy, propoxy, butoxy, hexyloxy and decyloxy radicals.

The term “cycloaliphatic radical” means, preferably, mono- or polycyclic radicals, such as the 1-methylcyclohexyl radical or the 1-adamantyl radical.

The term “salts of the naphthoic acid derivatives” means salts formed with a pharmaceutically acceptable base, in particular, an inorganic base, such as sodium hydroxide, potassium hydroxide and aqueous ammonia, or an organic base, such as lysine, arginine or N-methylglucamine, but also the salts formed with fatty amines, such as dioctylamine, aminomethylpropanol and stearylamine.

The term “esters of the naphthoic acid derivatives” means esters formed with pharmaceutically acceptable alcohols.

Preferably, the selection will be made, among the naphthoic acid derivatives included in the compositions according to the invention, of 6-[3-(1-adamantyl)4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid or 6-[3-(l -adamantyl)-4-hexyloxyphenyl]-2-naphthoic acid.

More preferably still, the retinoid compounds according to the invention are selected from among adapalene (6-[3-(1-adamantyl)-4-methoxyphenyl]-2-naphthoic acid), its salts and its esters.

The term “adapalene salts” means, in particular, the salts formed with a pharmaceutically acceptable base, in particular, inorganic bases, such as sodium hydroxide, potassium hydroxide and aqueous ammonia, or organic bases, such as lysine, arginine or N-methylglucamine.

The term “adapalene salts” also means the salts formed with fatty amines, such as dioctylamine, aminomethylpropanol and stearylamine.

Preferably, the retinoid compound is adapalene.

The anti-irritants according to the present invention are selected from among allantoin, EDTA, divalent strontium salts, divalent zinc salts, monovalent sodium salts and the hydrated derivatives thereof. The inclusion of these specific anti-irritants makes it possible to reduce the irritation caused by retinoids, in particular, adapalene.

The term “divalent strontium salts” means in particular, strontium nitrate, strontium chloride, strontium sulfide, strontium carbonate and strontium bromide. Preferably, the divalent strontium salts are strontium nitrate and strontium chloride hexahydrate.

The term “divalent zinc salts” means, in particular, zinc sulfate, zinc chloride, zinc carbonate and zinc citrate. Preferably, the divalent zinc salt is zinc sulfate.

The term “monovalent sodium salt” means, preferably, sodium choleate.

The term “hydrated derivatives” means, in particular, the abovementioned anti-irritant compounds hydrated by one or more molecules of water. Preferably, the hydrated derivatives are strontium chloride hexahydrate or strontium bromide hexahydrate.

Preferably, the anti-irritant compounds are selected from among strontium nitrate, allantoin, zinc sulfate, sodium choleate, strontium chloride hexahydrate and EDTA.

Preferably, the anti-irritant is allantoin or strontium nitrate.

In the compositions according to the invention, the concentration of retinoid compound is from 0.001% to 10% by weight, preferably from 0.01% to 5% by weight and more preferably from 0.05% to 2% by weight of the total weight of the composition. Herein, unless otherwise specified, it is understood that, when concentration intervals are given, they include the upper and lower limits of said interval.

Preferably, the concentration of retinoid compound is equal to 0.01%. Alternatively, the concentration of retinoid compound is preferably equal to 0.3%.

The concentration of anti-irritant compound is, for its part, from 0.01% to 10%, preferably from 0.1% to 7% by weight.

The compositions according to the present invention can be provided in all the formulation forms normally employed for topical application, in particular, in the form of aqueous, aqueous/alcoholic or oily dispersions, of dispersions of the lotion type, of aqueous, anhydrous or lipophilic gels, of emulsions with a liquid or semi-liquid consistency of the milk type, obtained by dispersion of a fatty phase in an aqueous phase (O/W) or vice versa (W/O), or of suspensions or emulsions with a soft, semi-liquid or solid consistency of the cream, cream gel, foam or ointment type, or of microemulsions, of microcapsules, of microparticles or of vesicular dispersions of ionic and/or nonionic type, or in the form of sprays.

Preferably, the compositions are provided in the form of a gel.

One skilled in this art will take care to select the excipients constituting the compositions according to the invention as a function of the formulation form desired and such that the advantageous properties of the composition according to the invention are retained.

In addition, the compositions according to the invention can, in particular, comprise one or more of the following ingredients:

a) one or more gelling agents or suspending agents,

b) one or more chelating agents other than EDTA,

c) one or more wetting agents,

d) one or more preservatives.

Exemplary gelling agents or suspending agents which can be included in the compositions according to the invention are carbomers marketed under the generic name of Carbopol®, carbomers said to be insensitive to electrolytes marketed under the trademark of Ultrez 10® or of Carbopol ETD by BF Goodrich, polysaccharides, with, as non-limiting examples, xanthan gum, such as Keltron T®, marketed by Kelco, guar gum, chitosans, cellulose and its derivatives, such as hydroxyethylcellulose, in particular, the product marketed under the trademark of Natrosol HHX 250® by Aqualon, and the copolymer of sodium acrylamide and of acrylamido-2-methylpropanesulfonate as a 40% dispersion in isohexadecane, and polysorbate 80, marketed under the trademark of Simulgel 600® by Seppic.

A preferred gelling agent is hydroxyethylcellulose, marketed, in particular, under the trademark Natrosol HHX 250®.

Exemplary chelating agents include diethylenetriaminepentaacetic acid (DTPA), ethylenediaminedi(o-hydroxyphenylacetic acid) (EDDHA), (2-hydroxyethyl)ethylenediaminetriacetic acid (HEDTA), ethylenediaminedi(o-hydroxy-p-methylphenylacetic acid) (EDDHMA) and ethylenediaminedi(5-carboxy-2-hydroxyphenylacetic acid) (EDDCHA).

Preferably, wetting agents are included, the role of which is to reduce the surface tension and to make possible greater spreading of the liquid, and exemplary thereof are compounds such as propylene glycol, dipropylene glycol, propylene glycol dipelargonate, lauroglycol and ethoxydiglycol, alone or as a mixture. Also, compounds may be included known for their role as emulsifiers, such as Tween 80, glyceryl monostearate & POE stearate, marketed under the trademark Arlacel 165FL® by Uniquema, polyoxyethylene (21) stearyl ether, marketed under the trademark Brij 721® by Uniquema, or Synperonics, with in particular, Synperonic PE/L62 (poloxamer 182) or Synperonic PE/L44 (poloxamer 124).

A preferred wetting agent is propylene glycol, Synperonic PE/L62 (poloxamer 182) or Synperonic PE/L44 (poloxamer 124).

Exemplary preservatives that can be included are benzoic acid and its derivatives with benzyl alcohol, benzalkonium chloride, sodium benzoate, bronopol, chlorhexidine, chlorocresol and its derivatives, ethyl alcohol, phenethyl alcohol, phenoxyethanol, potassium sorbate, diazolidinylurea, parabens, such as propylparaben or methylparaben, taken alone or as mixtures.

Preferred preservatives are the parabens and phenoxyethanol or benzalkonium chloride, alone or as mixtures.

The compositions according to the invention can also comprise one or more emulsifiers.

Surface-active emulsifiers are amphiphilic compounds which have a hydrophobic moiety possessing an affinity for the oil and a hydrophilic moiety possessing an affinity for the water, thus creating a connection from the two phases. Ionic or nonionic emulsifiers thus stabilize oil/water emulsions by being adsorbed at the interface and by forming lamellar layers of liquid crystals.

Exemplary preferred emulsifiers include the emulsifiers mentioned above for their property of wetting agents or lipophilic emulsifiers of glucate SS and glucamate SSE type.

The compositions of the invention can additionally comprise any additive normally used in the cosmetics or pharmaceutical field, such as neutralizing agents, sunscreens, antioxidants, fillers, electrolytes, colorants, normal inorganic or organic bases or acids, fragrances, essential oils, cosmetic active principles, moisturizing agents, vitamins, essential fatty acids, sphingolipids, self-tanning compounds, such as DHA, soothing and skin-protecting agents, propenetrating agents or a mixture of these. Of course, one skilled in the art will take care to choose this or these optional additional compounds and/or their amounts such that the advantageous properties of the composition according to the invention are not, or not substantially, detrimentally affected.

These additives can be present in the composition in a proportion of 0.001% to 20% by weight, with respect to the total weight of the composition.

The present invention also features administration of the subject compositions as described above as medicaments.

In particular, this invention features the formulation of the subject compositions as described above into medicaments useful for the treatment and/or prevention of dermatological conditions or afflictions related to a disorder of keratinization relating to cell differentiation and to cell proliferation, in particular, in treating acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne and acne medicamentosa.

Preferably, the present invention features formulation of a composition as described above into medicaments useful to prevent and/or treat acne vulgaris, whether regime or regimen.

Preferably, said compositions according to the invention are administered topically.

In addition, this invention also features the cosmetic application of the subject compositions in the treatment of skin having a tendency toward acne, in order to combat the greasy appearance of the skin or hair.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Solutions of Anti-Irritants

The anti-irritants used are formulated, unless otherwise indicated, in an ethanol/water (50:50) vehicle at the concentrations shown in the table below. The latter also shows, for each anti-irritant, the group treated in Example 2.

| | | Content in 50/50 |
|---|---|---|
| | | aqueous/alcoholic |
| Group | Anti-irritants | solution (%) |
| 4 | Enoxolone | 1.5 |
| 5 | Zinc sulfate | 0.5 |
| 6 | Potassium disodium salt of β-glycyrrhizic | 1.5 |
| | acid | |
| 7 | Sodium choleate | 2.5 |
| 8 | Strontium nitrate | 5.0 |
| 9 | Allantoin | 0.2 |
| 10 | Strontium chloride hexahydrate | 3.3 |
| 11 | Strontium chloride hexahydrate | 6.6 |
| 12 | EDTA | 3.0 |
| 13 | EDTA | 1.0 |

Groups 4 and 6 are used as negative controls in the studies which follow. This is because, as shown in the following studies, although being known as anti-irritants, the compounds used in these two groups (enoxolone and potassium disodium salt of β-glycyrrhizic acid) do not have an effect on the irritation due to retinoids.

### EXAMPLE 2

### Evaluation of the Effects of Various Anti-Irritants in Preventive Treatment Before Topical Application of Differin® Gel on the BALB/c Mouse; Tolerance Study:

The goal of the present study is to compare the irritant power of a reference gel, comprising 0.1% adapalene, when this treatment is preceded or not preceded by treatment with an anti-irritant.

The treatment consists of a daily topical application (20 μl) of anti-irritant, formulated in an aqueous/alcoholic vehicle (50% ethanol and 50% water by volume), on the internal face of the right ear of BALB/c mice divided into 15 groups (female mice approximately 9 weeks old), followed by a topical application (20 μl) of Differin® gel (reference gel comprising 0.1% adapalene) at the rate of one application of each formulation per day for 6 days.

The test products are:

Group 1: Untreated (controls)

Group 2: Differin® gel (reference gel)

Group 3: Ethanol/water solution (aqueous/alcoholic vehicle for the anti-irritants), followed by Differin® gel

Group 4: Enoxolone, followed by Differin® gel

Group 5: Zinc sulfate, followed by Differin® gel

Group 6: Potassium disodium salt of β-glycyrrhizic acid, followed by Differin® gel

Group 7: Sodium choleate, followed by Differing gel

Group 8: Strontium nitrate, followed by Differin® gel

Group 9: Allantoin, followed by Differin® gel

Group 10: 3.3% Strontium chloride hexahydrate, followed by Differin® gel

Group 11: 6.6% Strontium chloride hexahydrate, followed by Differin® gel

Group 12: 3% EDTA, followed by Differin® gel

Group 13: 1% EDTA, followed by Differin® gel

Evaluation is carried out by measurements of the thickness of the ear by means of the Oditest and by clinical observation of the animals from the 2^{nd} to 19^{th} day.

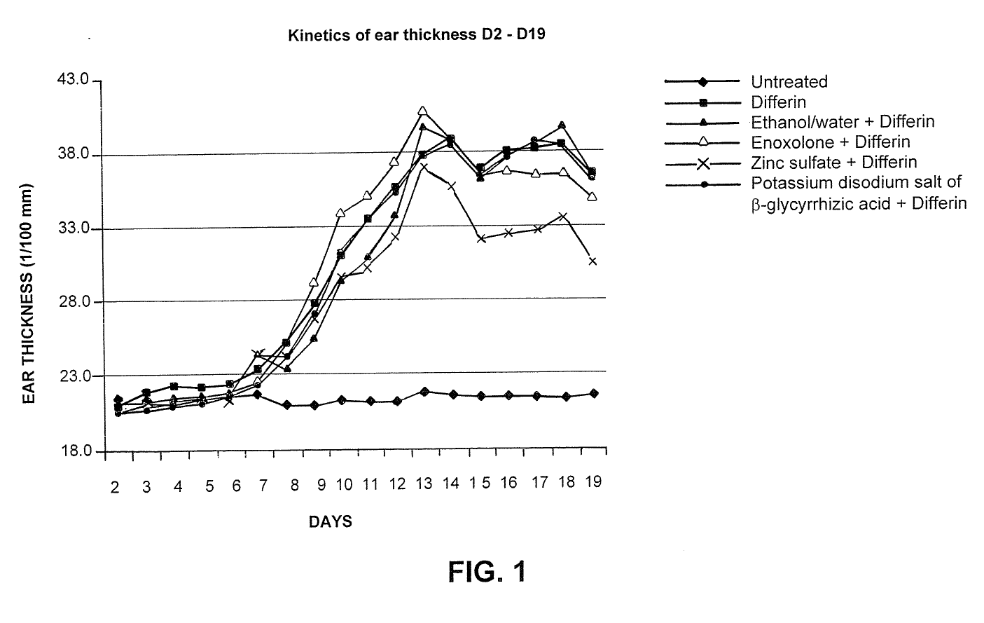

The results are represented in the table below and in FIGS. 1 to 3, where:

FIG. 1 is the kinetics of the mean thickness of the mouse ears from the 2^{nd} and 19^{th} days for groups 1 to 3 (references) and 4 to 6.

These kinetics show that the Differin® gel formulation alone is an irritant; the addition of the aqueous/alcoholic vehicle to this formulation does not change the degree of irritation (group 3).

Furthermore, a reduction in the irritation of the formulation is observed with zinc sulfate. On the other hand, enoxolone and the potassium disodium salt of β-glycyrrhizic acid have virtually no effect on the decrease in the irritation caused by Differin® gel.

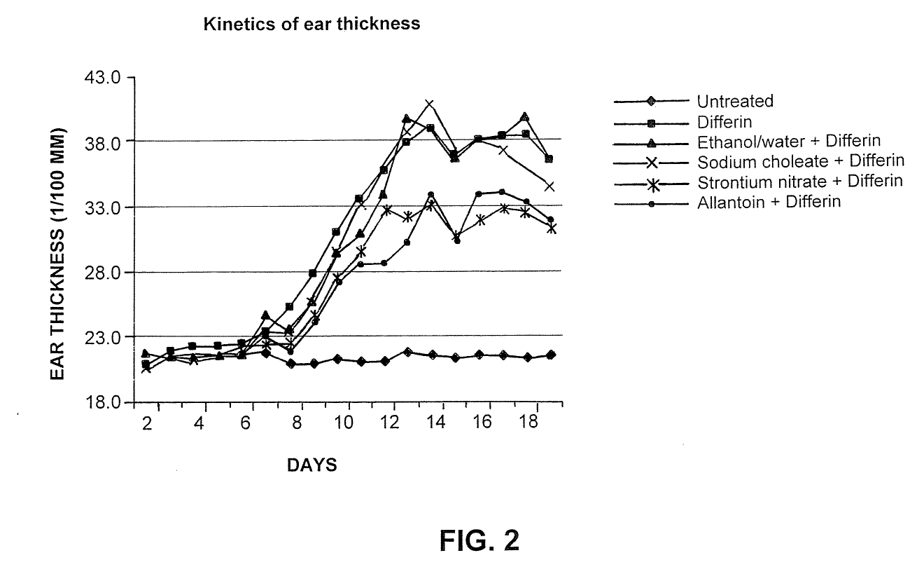

FIG. 2 is the kinetics of the mean thickness of the mouse ears from the 2^{nd} and 19^{th} days for groups 1 to 3 (reference) and 7 to 9.

These kinetics show, for group 7, that sodium choleate slightly reduces the irritation due to Differin® gel.

For their part, strontium nitrates and allantoin surprisingly reduce the irritation due to Differin® gel, in respective proportions of 37 and 40%.

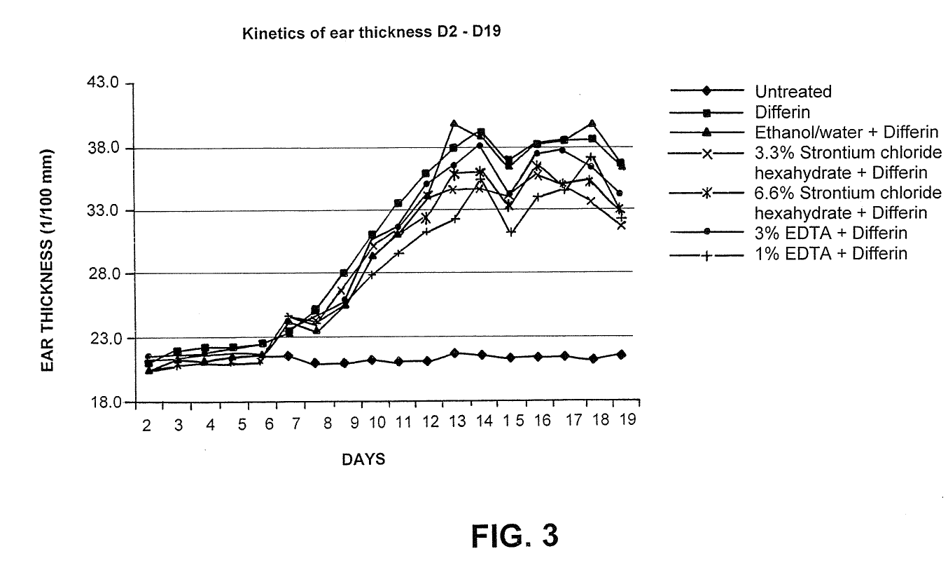

FIG. 3 is the kinetics of the mean thickness of the mouse ears from the 2^{nd} and 19^{th} days for groups 1 to 3 (references) and 10 to 13.

These kinetics, which are very similar for groups 10 to 13, show that strontium chloride hexahydrate and EDTA reduce the irritation due to Differin® gel by at least 10%.

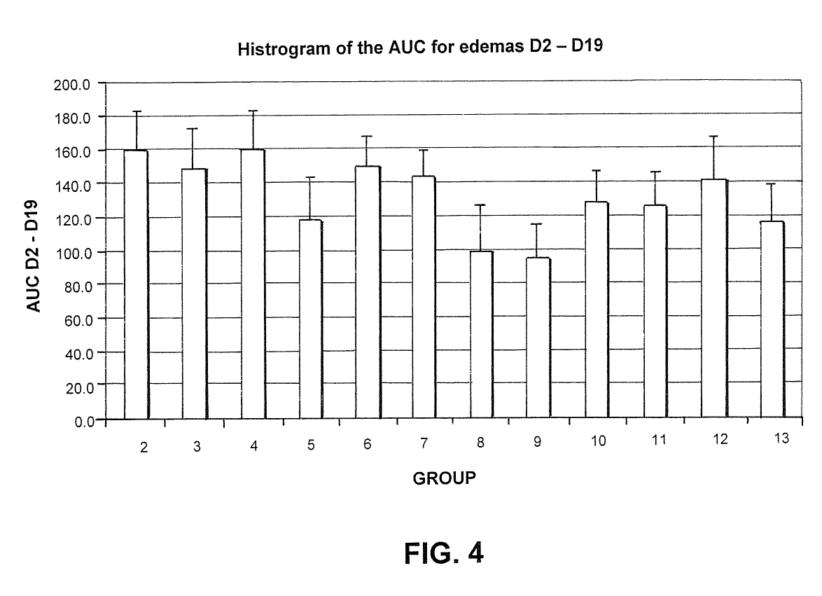

Summarizing table for the results of the areas under the curve (AUC) for the kinetics of the ear thicknesses (cf. FIG. 4 for the histograms).

| | AUC for | | % AUC | | |
|---|---|---|---|---|---|
| | edema D2- | | inhibition | | Student's |
| | D19 | | vs | p | t-test vs |
| Untreated | Mean | SEM | Differin | values | Differin |
| Differin | 158.6 | 24.4 | | | |
| Ethanol/water + Differin | 147.6 | 12.8 | 7.0 | 0.6989 | NS |
| Enoxolone + Differin | 159.0 | 23.8 | −0.3 | 0.9909 | NS |
| Zinc sulfate + Differin | 118.1 | 24.5 | 25.5 | 0.2755 | NS |
| Potassium disodium salt | 149.7 | 17.1 | 5.6 | 0.7727 | NS |
| of β-glycyrrhizic acid + | | | | | |
| Differin | | | | | |
| Sodium choleate + | 144.1 | 15.7 | 9.1 | 0.6307 | NS |
| Differin | | | | | |
| Strontium nitrate + | 99.8 | 26.1 | 37.1 | 0.1379 | NS |
| Differin | | | | | |
| Allantoin + Differin | 95.2 | 18.5 | 40.0 | 0.0720 | NS |
| 3.3% Strontium chloride | 127.8 | 24.9 | 19.4 | 0.4029 | NS |
| hexahydrate + Differin | | | | | |
| 6.6% Strontium chloride | 125.6 | 21.6 | 20.8 | 0.3407 | NS |
| hexahydrate + Differin | | | | | |
| 3% EDTA + Differin | 141.3 | 19.0 | 10.9 | 0.5909 | NS |
| 1% EDTA + Differin | 116.8 | 26.1 | 26.4 | 0.2759 | NS |

NS = Not Significant

The results of the study show that, after repeated topical applications of 20 μl of an anti-irritant solution, followed by 20 μl of Differin® gel, from D1 to D6 on the ear of the BALB/c mouse:

the Differin® gel formulation is an irritant;

the anti-irritants tested enoxolone and potassium disodium salt of β-glycyrrhizic acid do not have an effect on the irritation caused by Differin® gel;

the anti-irritants tested zinc sulfate, sodium choleate, strontium chloride hexahydrate and EDTA reduce the edema by 25%, 9%, 20% to 10% at least respectively;

the anti-irritants strontium nitrate and allantoin reduce the edema in a much more significant fashion (at least 37%).

This example shows that the anti-irritants do not all have the same effect with regard to the edema caused by Differin® gel and that only zinc sulfate, sodium choleate, strontium chloride hexahydrate, EDTA, strontium nitrate and allantoin are effective in reducing the irritation due to adapalene.

It should also be noted that no loss in weight is recorded during the study.

### EXAMPLE 3

Evaluation of the Comedolytic Activity of Differin® Gel Alone Compared with Differin® Gel in Combination With a Placebo Comprising an Anti-Irritant on the Rhino Mouse:

The goal of the present study is to compare the comedolytic activity of Differin® gel (reference gel comprising 0.1% adapalene) whether this treatment is preceded or not preceded by a treatment with an anti-irritant.

The treatment consists of a daily topical application of an aqueous/alcoholic vehicle (ethanol/water 50:50) comprising an anti-irritant (strontium nitrate or allantoin), followed 30 minutes later by an application of Differin® gel, on the skin of the back of the RHINO FVB/N RJ-hr^{rth} (Rhino) mouse for 18 days.

The test products are:

Group 1: Differin® gel alone

Group 2: Aqueous/alcoholic vehicle, followed by Differin® gel placebo

Group 3: Aqueous/alcoholic vehicle, followed by Differin® gel

Group 4: Strontium nitrate, followed by Differin® gel

Group 5: Allantoin, followed by Differin® gel.

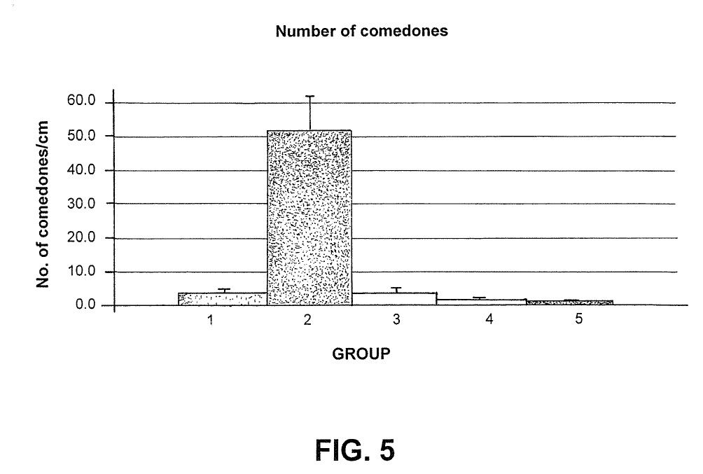

FIG. 5 shows the results of the counting of the number of comedones per centimeter [cm] on the back of the Rhino mice after 18 days of topical treatment for the 5 groups mentioned above.

The results of the study show that skin treated with placebos (group 2) exhibits a high number of comedones per centimeter, of from 51 and 60. Skin treated with Differin® gel alone or preceded by a placebo (groups 1 and 3) exhibits a comparable and low number of comedones per centimeter, of from 3 and 5.

Skin treated beforehand with an anti-irritant (allantoin or strontium nitrate) exhibits a statistically lower number of comedones per centimeter than groups 1 and 3 (number of comedones from 1 and 2). However, given the abnormally high comedolytic activity of group 1, this significant difference is not regarded as relevant biologically.

It thus emerges from FIG. 5 that the combination of an allantoin or strontium nitrate anti-irritant with Differin® gel in a split treatment does not reduce the comedolytic activity of Differin® gel alone.

Finally, after 18 days of topical treatment, it should be noted that the animals do not exhibit a loss in weight.

This overall study demonstrates that the application of a specific anti-irritant before the treatment with Differin® gel does not reduce the comedolytic activity of adapalene.

### EXAMPLE 4

### Formulations of Gel Type Comprising 0.1% Adapalene and Anti-Irritants:

| Ingredients | Formula A | Formula B |
|---|---|---|
| Adapalene | 0.1% | 0.1% |
| Purified water | 80.0% | 67.5% |
| Allantoin | 0.2% | — |
| Strontium nitrate | — | 5.0% |
| Titriplex III | 0.2% | 0.2% |
| Natrosol 250 HHX Pharm | 2.0% | 2.0% |
| Propylene glycol | 4.0% | 4.0% |
| Synperonic PE/L62 | 0.2% | 0.2% |
| Phenoxyethanol | 1.0% | 1.0% |
| Purified water | q.s. for 100% | q.s. for 100% |

### EXAMPLE 5

### Study of Tolerance of the Formulations of Example 4:

A study of tolerance is carried out according to the protocol of Example 2 with the formulations of Example 4. However, the present case, in contrast to Example 2, relates to a treatment which is not split since the. adapalene and the anti-irritant are present in the same formulation.

The results of the areas under the curve (AUC) for the kinetics of ear thickness from days 2 and 19 are reported in the following table:

| | | | % | Student's | % |
|---|---|---|---|---|---|
| | AUC D2-D19 | | Increase | t-test | Inhibition |
| | | Standard | vs | vs | vs Differin |
| | Mean | deviation | placebo | placebo | gel |
| Differin | 358.0 | 3.8 | | | |
| placebo | | | | | |
| Differin gel, | 519.5 | 25.8 | 45.1 | — | |
| 0.1% | | | | | |
| Formula A | 453.8 | 11.1 | 28.4 | — | 12.6 |
| Formula B | 412.5 | 15.0 | 14.9 | — | 20.6 |

### Conclusions of the Study:

0.1% Differin® gel increases the area under the curve by 45% with respect to the placebo gel.
The formulations with anti-irritant increase the area under the curve with respect to the placebo gel according to the following order: formula A>formula B.
With respect to 0.1% Differin® gel, formula B is less irritating by 20%.
Strontium nitrate (formula B) appears to be the most effective anti-irritant. These results confirm those of Example 2, where the anti-irritants have been evaluated in the split treatment, namely, before application of 0.1% Differing gel.
Allantoin (formula A) formulated in a gel appears to be relatively ineffective in reducing the irritation due to adapalene.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/pharmaceutical composition comprising at least one retinoid compound selected from the group consisting of all trans retinoic acid, isotretinoin, motretinide, naphthoic acid compounds having the structural formula (I) and the salts and esters thereof: 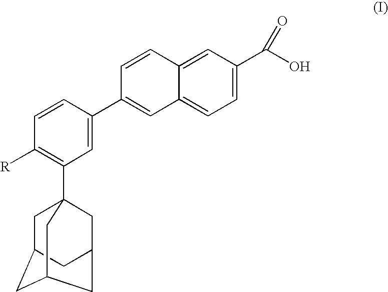
where R is a hydrogen atom, a hydroxyl radical, a linear or branched alkyl radical having from 1 to 4 carbon atoms, an alkoxy radical having from 1 to 10 carbon atoms or a substituted or unsubstituted cycloaliphatic radical, and at least one anti-irritant compound selected from the group consisting of allantoin, divalent strontium salts, divalent zinc salts, monovalent sodium salts and the hydrated derivatives thereof, formulated into a topically applicable, physiologically acceptable medium therefor.

2. The cosmetic/pharmaceutical composition as defined by claim 1, devoid of any depigmenting agent other than said at least one retinoid.

3. The cosmetic/pharmaceutical composition as defined by claim 1, wherein said at least one retinoid has the formula (I) and R is a methyl, ethyl, propyl or butyl radical.

4. The cosmetic/pharmaceutical composition as defined by claim 1, wherein said at least one retinoid has the formula (I) and R is a methoxy, ethoxy, propoxy, butoxy, hexyloxy or decyloxy radical.

5. The cosmetic/pharmaceutical composition as defined by claim 1, wherein said at least one retinoid has the formula (I) and R is the 1-methylcyclohexyl radical or the 1-adamantyl radical.

6. The cosmetic/pharmaceutical composition as defined by claim 1, wherein said at least one retinoid compound is selected from the group consisting of adapalene, 6-[3-(1-adamantyl)-4-hydroxyphenyl]-2-naphthoic acid, 6-[3-(l -adamantyl)-4-decyloxyphenyl]-2-naphthoic acid and 6-[3-(1-adamantyl)-4-hexyloxyphenyl]-2-naphthoic acid.

7. The cosmetic/pharmaceutical composition as defined by claim 1, wherein said at least one retinoid compound is adapalene.

8. The cosmetic/pharmaceutical composition as defined by claim 1, wherein said at least one anti-irritant compound is strontium nitrate or strontium chloride.

9. The cosmetic/pharmaceutical composition as defined by claim 1, wherein said at least one anti-irritant compound is strontium chloride hexahydrate or strontium bromide hexahydrate.

10. The cosmetic/pharmaceutical composition as defined by claim 1, wherein said at least one anti-irritant compound is allantoin, zinc sulfate, or sodium choleate.

11. The cosmetic/pharmaceutical composition as defined by claim 1, formulated as a gel.

12. The cosmetic/pharmaceutical composition as defined by claim 1, said at least one retinoid compound comprising from 0.001% to 10% by weight of the total weight thereof.

13. The cosmetic/pharmaceutical composition as defined by claim 9, wherein the concentration of said at least one retinoid compound is about 0.1%.

14. The cosmetic/pharmaceutical composition as defined by claim 9, wherein the concentration of said at least one retinoid compound is about 0.3%.

15. The cosmetic/pharmaceutical composition as defined by claim 1, wherein the concentration of said at least one anti-irritant compound ranges from 0.01% to 10% by weight of the total weight thereof.

16. The cosmetic/pharmaceutical composition as defined by claim 1, formulated as a medicament.

17. A regime or regimen for the treatment and/or prevention of a dermatological condition or affliction related to a disorder of keratinization relating to cell differentiation and to cell proliferation, comprising topically applying onto the affected skin area of an individual in need of such treatment, a thus effective amount of the cosmetic/pharmaceutical composition as defined by claim 1.

18. A regime or regimen for the treatment of acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne or acne medicamentosa, comprising topically applying onto the affected skin area of an individual in need of such treatment, a thus effective amount of the cosmetic/pharmaceutical composition as defined by claim 1.

19. The regime or regimen as defined by claim 15, comprising the treatment and/or prevention of acne vulgaris.

20. A regime or regimen for the treatment of skin having a tendency toward acne or for combating the greasy appearance of the skin or hair, comprising topically applying onto the affected skin area or hair of an individual in need of such treatment, a thus effective amount of the cosmetic/pharmaceutical composition as defined by claim 1.
